(19) United States
(12) Patent Application Publication
(10) Pub. No.: US 2013/0079700 A1
(43) Pub. Date: Mar. 28, 2013

(54) COMPOSITE PROSTHETIC SHUNT DEVICE

(71) Applicant: Zeus Industrial Products, Inc., Orangeburg, SC (US)

(72) Inventors: Robert L. Ballard, Orangeburg, SC (US); Bruce L. Anneaux, Lexington, SC (US); Sabrina D. Puckett, Lexington, SC (US)

(73) Assignee: Zeus Industrial Products, Inc., Orangeburg, SC (US)

(21) Appl. No.: 13/625,548

(22) Filed: Sep. 24, 2012

Related U.S. Application Data
(60) Provisional application No. 61/538,402, filed on Sep. 23, 2011.

(51) Int. Cl. A61M 1/00; B29C 65/70; B32B 37/26

(52) U.S. Cl. 604/8; 156/294; 264/263

(57) ABSTRACT

In accordance with certain embodiments of the present disclosure, a composite prosthetic device is described. Generally, the device comprises at least one layer of ePTFE, at least one thermoplastic elastomeric component, and a frame. In certain aspects, the thermoplastic elastomeric component penetrates the microstructure of the at least one layer of ePTFE, providing a means for varying the porosity of the ePTFE.

### CROSS-REFERENCE TO RELATED APPLICATIONS

This application claims priority to international application number PCT/US2012/56757, filed on Sep. 21, 2012, and to U.S. provisional patent applicatcion No. 61/538,402, filed Sep. 23, 2011, which are both incorporated herein by reference in their entireties.

### BACKGROUND

Dialysis treatment of patients suffering from kidney failure requires that their blood be withdrawn and passed through a dialysis machine. The process is known as hemodialysis and must be performed regularly. Hemodialysis requires the insertion of a large bore needle into a patient's vein to effect the removal and recycling of the blood. Repeated insertion of such a needle is damaging to the vein and is thus not a long term solution. Prosthetic arteriovenous (AV) shunt grafts (that provide a connection between a vein and an artery) have been developed that provide an access site for insertion of the needle.

Prosthetic shunt grafts are usually constructed of polymeric materials. However, it is important for the polymeric shunt material to meet certain requirements which provides a major challenge. Such challenges namely include biocompatibility, the ability to be attached and anchored at a location within the body, the ability to be readily penetrated by a needle, but have a self-sealing capability after removal of the needle, and to have sufficient resiliency (e.g., such that the shunt can be found, yet be compliant and flexible enough to allow recovery of the shunt upon removal of the needle.

The successful use of extruded tubes of expanded polytetrafluoroethylene (ePTFE) as synthetic implantable vascular prostheses or tubular grafts, designed in particular for such applications, is well-known and documented. ePTFE, validated through significant clinical studies, is particularly suitable as a vascular prosthesis and/or tubular graft material as it exhibits superior biocompatibility and can be mechanically manipulated to form a well-defined porous microstructure known to promote endothelialization and thus attachment and anchoring within the body. PTFE has been proven to exhibit a low thrombogenic response in vascular applications. The microporous structure, formed of nodes and fibrils, allows natural tissue ingrowth and endothelialization when implanted in the vascular system. This contributes to long term healing and patency of the tubular graft. When seeded or infused with a bio-active agent, healing rate, tissue proliferation, and endothelialization can all be manipulated.

U.S. Pat. No. 6,436,135 to Goldfarb describes the microstructure of a synthetic vascular prostheses or tubular graft formed of ePTFE as being categorized by a fibrous state which is further defined by irregularly spaced nodes interconnected by elongated fibrils or microfibers. The method and techniques for creating this type of structure have been known for more than three decades. The distance between the node surfaces that is spanned by the fibrils is defined as the internodal distance (IND). A tubular graft having a specific range of IND enhances tissue ingrowth and cell endothelialization, as the tubular graft is inherently porous. The IND range is generally small enough to prevent transmural blood flow and thrombosis, but not less than the maximum dimension of the average red blood cell, between 6 μm and 80 μm.

There are numerous examples of microporous ePTFE tubular vascular prostheses or tubular grafts. The porosity of an ePTFE vascular prosthesis or tubular graft is controlled by the mechanical formation of the IND or the microporous structure of the tube. IND with the defined structure referenced can produce results of tissue ingrowth as well as cell endothelialization along the inner and/or outer surface of the vascular prosthesis or tubular graft. Recently, studies have shown that improved grafts and/or stent-grafts with new and/or improved characteristics may be made by the introduction of multilayer composites into the devices. Namely, U.S. Pat. No. 8,262,979 to Anneaux et al., incorporated by reference herein, describes examples of such prosthetic devices.

Stents are commonly used to restore and maintain body passages, such as blood vessels. Often, biocompatible materials, including grafts, can be provided on the inner and/or outer surfaces of the stent to reduce reactions associated with contact of the stent with the body. Another potential use for stent-grafts is in the area of “shunts”. A shunt, as used herein, is a stent that is used to connect two blood vessels (e.g., an artery and a vein) and can be used for the interjection of fluids into the body or the removal of blood or blood components from the body. An example of such a device would be a shunt used for dialysis. There is a need for a biocompatible device having sufficient physical properties to be utilized in this capacity.

### SUMMARY OF THE INVENTION

In accordance with certain embodiments of the present disclosure, a description of a prosthetic device is provided. The device comprises a frame (e.g., a stent), at least one ePTFE layer, and at least one thermoplastic elastomer. In certain embodiments, the device is configured such that ePTFE layers make up the inner diameter (ID) and outer diameter (OD) of the device. In such embodiments, the frame is in the interior of the device. The thermoplastic elastomer component typically provides adhesion between two or more of the device components. For example, in certain embodiments, the frame can be imbedded within the thermoplastic elastomer component; the thermoplastic elastomer component can further adhere to and/or penetrate one or more of the ePTFE layers.

In some embodiments, the thermoplastic elastomer component provides a means to control the porosity/permeability of the device. For example, in certain embodiments, the thermoplastic elastomer may completely fill the pores (i.e., the spaces between the nodes/fibrils) of the ePTFE layer or layers.

In one aspect of the invention is provided a composite prosthetic shunt comprising: an inner lumen; a first tubular layer of expanded polytetrafluoroethylene (ePTFE) having nodes and fibrils around the inner lumen; a tubular frame imbedded in a thermoplastic elastomer positioned around and overlying the first tubular layer of ePTFE; and a second tubular layer of ePTFE having nodes and fibrils positioned around and overlying the tubular frame imbedded in a thermoplastic elastomer; wherein the thermoplastic elastomer penetrates at least about 50% of the spaces between the nodes and fibrils of at least one of the first and second tubular layers of ePTFE. The thermoplastic elastomer can vary; in certain embodiments, the thermoplastic elastomer comprises polyurethane.

Thus, in one aspect of the invention is provided a composite prosthetic shunt comprising: an inner lumen; a first tubular layer of expanded polytetrafluoroethylene (ePTFE) having nodes and fibrils around the inner lumen; a tubular frame imbedded in polyurethane positioned around and overlying the first tubular layer of ePTFE; and a second tubular layer of ePTFE having nodes and fibrils positioned around and overlying the tubular frame imbedded in polyurethane; wherein the polyurethane penetrates at least about 50% of the spaces between the nodes and fibrils of at least one of the first and second tubular layers of ePTFE.

In some embodiments, the average cross-sectional thickness of the shunt wall is between about 0.25 mm and about 0.51 mm. The shunt walls can, in certain embodiments, exhibit an average porosity of less than about 20%, such as an average porosity of about 0%. In some embodiments, the thermoplastic elastomer penetrates at least about 50% of the spaces between the nodes and fibrils of both the first and second tubular layers of ePTFE, at least about 80% of the spaces between the nodes and fibrils of at least one of the first and second tubular layers of ePTFE, or at least about 80% of the spaces between the nodes and fibrils of both the first and second tubular layers of ePTFE.

In certain embodiments, the composite prosthetic shunt exhibits a radial force such that after the shunt is compressed to close the inner lumen for 48 hours, the shunt fully reopens when the compression is removed. For example, a shunt having a lumen diameter of 4 mm may be compressed to close the lumen and, when compression is removed, a lumen having a diameter of at least 3 mm is obtained within 30 seconds after compression is removed. In some embodiments, the shunt exhibits an opening force of greater than about 200 grams, such as about 200 to about 300 grams.

In some embodiments, the composite prosthetic shunt exhibits no substantial decrease in performance after being compressed to close the inner lumen and then opened about 2,000 times or more or about 3,000 times or more. In this context, “no substantial decrease in performance” can, in certain embodiments, be evidenced by one or more of: no significant change in inside or outside dimensions of the shunt; no observable wear or deformation; no significant change in the recovery force of the shunt; and no significant loss of particulate material from the shunt.

In another aspect of the invention is provided a hemoaccess valve system comprising a composite prosthetic shunt as described herein. The system may comprise various components in addition to the composite prosthetic shunt and can be used in hemodialysis.

In a further aspect of the invention is provided a method for making a composite prosthetic shunt, comprising: applying a thermoplastic elastomeric sheet or tube to a construct comprising a tubular frame overlying a first ePTFE tubular structure; applying a second ePTFE tubular structure overlying the thermoplastic elastomeric sheet or tube to form a layered composite; compressing the layered composite; and heating the layered composite such that the thermoplastic elastomer penetrates at least about 50% of the spaces between the nodes and fibrils of at least one of the first and second tubular layers of ePTFE.

For example, in certain aspects, the invention provides a method for making a composite prosthetic shunt, comprising: applying a polyurethane sheet or tube to a construct comprising a tubular frame overlying a first ePTFE tubular structure; applying a second ePTFE tubular structure overlying the polyurethane sheet or tube to form a layered composite; compressing the layered composite; and heating the layered composite such that the polyurethane penetrates at least about 50% of the spaces between the nodes and fibrils of at least one of the first and second tubular layers of ePTFE.

In certain embodiments, the compressing and heating steps are conducted at the same time. The heating step is, for example, conducted at a temperature at or above the melting temperature of the thermoplastic elastomer. In some embodiments, the compressing step comprises wrapping the layered composite with a compression wrap.

In accordance with certain specific embodiments of the present disclosure, a device is provided which includes an ePTFE (biax) tube, tubular frame, polyurethane tube, and a second ePTFE (biax) tube covering the whole. In construction, the tubular frame, such as a stent, is positioned over a tubular biax ePTFE polymer. A polyurethane tube is then placed around the stent frame followed by another tubular biax ePTFE tube over the whole to form the prosthetic device. The structure is compressed and heated and the polyurethane slightly melted to provide adhesion between the biax layers and the stent.

Other features and aspects of the present disclosure are discussed in greater detail below.

### BRIEF DESCRIPTION OF THE DRAWINGS

A full and enabling disclosure, including the best mode thereof, directed to one of ordinary skill in the art, is set forth more particularly in the remainder of the specification, which makes reference to the appended figures in which:

FIG. 1 illustrates a cross sectional view of a prosthetic device configuration in accordance with certain embodiments of the present disclosure;

FIG. 2 illustrates the inner portion (ID) of a construct including a radially expanded fully sintered ePTFE biax tube in accordance with certain embodiments of the present disclosure;

FIG. 3 illustrates the outer portion (OD) of a construct including a radially expanded fully sintered ePTFE biax tube in accordance with certain embodiments of the present disclosure;

FIG. 4 illustrates an ePTFE/frame/PU/ePTFE construct in accordance with certain embodiments of the present disclosure;

FIG. 5 illustrates a cross-sectional view of an ePTFE/frame/PU/ePTFE construct indicating adhesion of the layers after heating (no delamination) in accordance with certain embodiments of the present disclosure;

FIG. 6 illustrates a cross-sectional image of an ePTFE/frame/PU/ePTFE construct, showing the melting of PU into the ePTFE biax layers in accordance with certain embodiments of the present disclosure; and

FIG. 7 illustrates cross-sectional images of two embodiments of ePTFE/frame/PU/ePTFE constructs.

### DETAILED DESCRIPTION

Reference now will be made in detail to various embodiments of the disclosure, an example of which is set forth below. The example is provided by way of explanation of the disclosure, and is not intended to be limiting. In fact, it will be apparent to those skilled in the art that various modifications and variations can be made based upon the present disclosure without departing from the scope or spirit of the disclosure. For instance, features illustrated or described as part of one embodiment can be used within another embodiment to yield further embodiments. Thus, it is intended that the present disclosure covers such modifications and variations as come within the scope of the appended claims and their equivalents.

The present invention provides tubular prosthetic devices (also referred to herein as “tubular vascular prostheses” and/or “tubular grafts”) comprising one or more layers of expanded polytetrafluoroethylene (also referred to herein as “expanded PTFE” or “ePTFE”), a frame (e.g., stent), and a suitable nonporous elastic polymeric material (thermoplastic elastomeric component) such as a polyamide, polyurethane (PU), polyester, fluorinated ethylene propylene (FEP), or the like. It is to be understood that, in addition to the general structures described herein, the present disclosure is intended to encompass devices having one or more additional layers of varying chemical makeup within the composite structure or overlying the composite structure.

The composite devices described in the present disclosure can, in certain embodiments, offer a number of advantages over conventional processes and devices including, but not limited to: 1) the ability to incorporate layers with different (e.g., vastly different) pore structures and sizes, wherein these different structural layers can be used to manipulate mechanical properties, cellular proliferation, cellular permeability, fluid permeability, adhesion to a structural frame, and/or incorporation of one or more active therapeutic components; 2) the ability to make a composite construction with different components (e.g., vastly different components) enabling a broader range of therapeutic uses and structures; 3) improved bonding of ePTFE layers to structural frames and to other layers of the construct; and/or 4) the ability to coat various complex geometries that otherwise could not be covered with ePTFE or other materials alone.

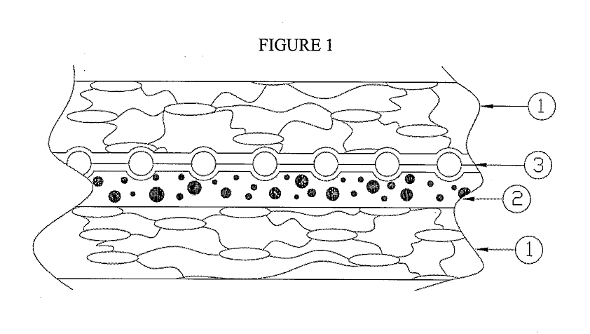

Advantageously, in certain embodiments, a tubular prosthetic device is provided wherein the inner diameter (ID), outer diameter (OD), or both the ID and the OD comprise ePTFE layers. In such embodiments, the stent or frame is in the interior of the device. Referring to FIG. 1, a cross-section of an exemplary composite device in accordance with the present disclosure is illustrated. The device includes an ePTFE biax layer 1, a thermoplastic elastomeric component 2, a frame 3, and another ePTFE biax layer 1.

Expanded PTFE generally exhibits a microstructure consisting of solid nodes interconnected by fine, highly oriented fibrils. The expanded PTFE nodes and fibrils provide unique biocompatible porous structures. The microstructure of the material can be tailored, in some cases, to provide a matrix for cellular attachment and ingrowth. Expanded PTFE can be designed and tailored to enhance, inhibit or retard the migration of endothelium during the early phase of healing. As an example, ePTFE microstructure with an internodal distance (IND) of about 10 μm to about 20 μm permits very little transmural cellular ingrowth. Optimal INDs for cellular ingrowth ranges between about 20 μm and about 80 μm. Studies have shown that INDs of greater than about 120 μm have been associated with reduced ingrowth and poor neointima adhesion based on the smaller surface area available for cellular adhesion and locomotion.

According to the present invention, the properties (e.g., pore size, pore structure, internodal distance (IND), and porosity) of the ePTFE used to construct the composite device can vary. Further, the one or more layers of ePTFE within a given composite device can have the same properties or can have different properties. Due to the construction of the composites, the pore size, pore structure, IND, and porosity can vary from layer to layer within the cross section of the composite, depending on the construction. An example would be an asymmetrical construction where pores change in size from large to small based on layer evaluations from surface to surface throughout the medium.

In certain embodiments, the ePTFE pore size (as defined by ASTM F316, incorporated by reference herein), can range from about 0.05 μm to about 50 μm, such as from about 0.1 μm to about 20 μm, or from about 0.2 μm to about 10 μm (e.g., from about 1 μm to about 3 μm). Advantageously, according to the invention, ePTFE with any IND value can be employed. For example, in certain embodiments, the ePTFE IND can be about 0.1 μm to about 200 μm (e.g., about 10 μm to about 50 μm, such as about 20 μm to about 40 μm). The porosity of the ePTFE used to construct the device can, in certain embodiments, range from about 20% to about 90% (it is noted that this porosity will, in some embodiments, change following the heating/compression steps described herein, such that the final composite device exhibits a lower ePTFE porosity).

Frames that can be incorporated within the composite devices described herein can take various forms, including but not limited to, stents, occlusion coils or frames, regenerative medicine scaffolds, structural reinforcements, pacing or monitoring leads, tissue anchors or tacks, biological stimulation devices, biomimetic implants, signal receivers or transmitters, orthopedic fixation devices, or any other metallic, polymeric, ceramic or other therapeutic devices. The frame can be, for example, a metal, ceramic, or polymeric frame. One exemplary material is nitinol. The frame in some embodiments is a stent, which is a tubular device commonly used to restore and/or maintain a body passage, such as a blood vessel.

The frame within the composite device generally serves to increase the radial strength of the overall construction and can also promote recovery during deployment of the construction. In certain embodiments of the present disclosure, the frame is a stent. The stent frame provides a structural backbone within the structure, which can prevent suture wall tear out.

The thermoplastic elastomeric component is beneficially used to adhere the stent or frame to the one or more ePTFE layers. The thermoplastic elastomeric component of the devices disclosed herein can vary, but is generally any polymeric material having a low porosity and/or liquid permeability. In certain embodiments, the thermoplastic elastomeric component is polyurethane (PU). PU, by nature, is a flexible, highly resilient polymer due to its chemical properties, in particular, its low glass transition temperature. Therefore, PU is ideal for applications such as prosthetic shunt grafts, where elasticity is needed to provide both resiliency and self-sealing capabilities. PU, when used as a film, has a low porosity (<0.5 μm) that will also prevent ingrowth and hence can serve as an impermeable layer according to the present invention when needed.

Advantageously, the thermoplastic elastomeric component is sandwiched within the wall of the device. In certain embodiments, the thermoplastic elastomeric component flows through the frame and adheres the frame to an ePTFE layer on the interior of the frame and/or to an ePTFE layer on the exterior of the frame.

Not only can the thermoplastic elastomeric component provide adhesion, but it may also, in some embodiments, modify the physical properties (e.g., the porosity and/or permeability) of the ePTFE on the ID and/or OD of the device. The thermoplastic elastomeric component can, in certain embodiments, penetrate one or both of the ePTFE layers to some degree, filling at least some of the pores (i. e., the spaces between the nodes/fibrils comprising the ePTFE microstructure). The heating and compression steps that effect the adhesion and penetration of the thermoplastic elastomer component are described further herein.

The thermoplastic elastomeric component can penetrate the pores (spaces between the nodes and fibrils) of the ePTFE on the ID and/or OD of the device in varying amounts. For example, in certain embodiments, the thermoplastic elastomeric component fills at least about 50% of the spaces between the nodes and fibrils of at least one of the ePTFE layers on the ID and OD of the device. In exemplary embodiments, the thermoplastic elastomer can fill at least about 60%, at least about 70%, at least about 80%, at least about 90%, at least about 95%, at least about 98%, at least about 99%, or about 100% (including 100%) of the spaces between the nodes and fibrils of the ePTFE on the ID, the ePTFE on the OD, or both the ePTFE on the ID and the ePTFE on the OD of the device. It is noted that penetration/filling can result in the blockage of one or more of the pores of the ePTFE layer(s). The depth of penetration of the thermoplastic elastomer component into the ePTFE layer(s) can vary. In some embodiments, the thermoplastic elastomer component penetrates the ePTFE to a depth of between about 5% and about 100% the thickness of the ePTFE. In some embodiments, the thermoplastic elastomer may not penetrate the entire thickness of the ePTFE layers and accordingly, there may be some degree of node/fibril structure on the surface (ID or OD) of the device.

As such, the thermoplastic elastomer component may reduce the porosity and/or permeability of the ePTFE. As such, the overall porosity of the wall of a device according to the invention may be as low as 0% (where the thermoplastic elastomer penetrates and completely fills the pores of the ePTFE on the ID, OD, or both ID and OD). The porosity of the composite device is advantageously close to 0% (e.g., less than about 5%, less than about 2.5%, less than about 1%, less than about 0.5%, less than about 0.1%, less than about 0.01%, less than about 0.001%, or about 0% (including 0%)).

Accordingly, the composite devices disclosed herein can be described as comprising a thermoplastic polymer that penetrates, to some degree, the microstructure of the one or more ePTFE layers.

The devices described herein can, in some embodiments, include one or more bioactive agents. Examples of bioactive agents that can be utilized in connection with the devices of the present disclosure include, but are not limited to, antibiotics, antifungals and antivirals such as erythromycin, tetracycline, aminoglycosides, cephalosporins, quinolones, penicillins, sulfonamides, ketoconazole, miconazole, acyclovir, ganciclovir, azidothymidine, vitamins, interferon; anticonvulsants such as phenytoin and valproic acid; antidepressants such as amitriptyline and trazodone; antiparkinsonism drugs; cardiovascular agents such as calcium channel blockers, antiarythmics, beta blockers; antineoplastics such as cisplatin and methotrexate, corticosteroids such as dexamethasone, hydrocortisone, prednisolone, and triamcinolone; NSAIDs such as ibuprofen, salicylates indomethacin, piroxicam; hormones such as progesterone, estrogen, testosterone; growth factors; carbonic anhydrase inhibitors such as acetazolamide; prostaglandins; antiangiogenic agents; neuroprotectants; neurotrophins; growth factors; cytokines; chemokines; cells such as stem cells, primary cells, and genetically engineered cells; tissues; and other agents known to those skilled in the art.

Typical construction of multiple layers may produce devices having wall thicknesses ranging from about 0.0025 mm to about 6.5 mm at widths of about 0.08 cm to about 30 cm. In certain exemplary embodiments, the composite device wall thickness (following heating and compression) is between about 0.01 inch and 0.05 inch (about 0.25 mm to about 1.3 mm), e.g., about 0.015 inch (about 0.38 mm) The individual layers can have thicknesses that vary, e.g., from about 0.0025 mm to about 6.5 mm For example, in certain embodiments, the ePTFE layers are each between about 0.01 inch and about 0.02 inch (about 0.25 mm to about 0.5 mm), such as between about 0.012 inch and about 0.013 inch (about 0.30 mm to about 0.33 mm) thick. In certain embodiments, the thermoplastic elastomeric component is initially provided in the faun of a material that is between about 0.001 inch and 0.01 inch (about 0.025 mm to about 0.25 mm) (e.g., about 0.005 inch (about 0.13 mm) thick prior to heating and compression. Final material size varies greatly as the composites can be produced as sheets or tubes at continuous roll lengths.

The properties and characteristics of the composite devices disclosed herein are the result of a compilation of the properties of the frame, thermoplastic elastomer, and ePTFE membrane layers. In certain embodiments, the ePTFE within the device has controlled fiber, node, and fibril sizes and can be manipulated mechanically, such as to improve bond strength, elongation properties, and tensile strengths in the final composite. In certain embodiments, even where the thermoplastic elastomeric component flows and fills in some percentage of the pores in one or both of the ID ePTFE layer and the OD ePTFE layers, one or both of these layers may still exhibit some degree of porosity. For example, in one specific embodiment, the OD of a composite device can exhibit some degree of porosity so as to encourage cell growth on the OD of the device. Advantageously, the ID generally exhibits little to no porosity so as to discourage cell growth on the ID of the device (e.g., to prevent biofouling of the ID).

Although the devices described herein can be used independently (e.g., as stents to restore and/or maintain a body passage, such as a blood vessel), in certain embodiments, they may be incorporated within other implantable systems. For example, one representative application for the devices described herein is as a component of a hemoaccess valve system. In such systems, a shunt (such as the device described herein) is employed in combination with a valve, such that the flow of blood through the device can be turned on when the patient needs vascular access for dialysis and can be turned off when the patient is not in dialysis. In this type of system, it may be possible to restore noimal blood flow to the artery and vein at the implantation site when the patient is not in dialysis. In certain embodiments, the valve can be used to close off the device and, optionally, allow it to be flushed and/or filled with saline until the patient's next dialysis session.

The devices of the present invention can advantageously operate under typical blood flow pressures (including high blood flow pressures, e.g., about 175 mm Hg). In preferred embodiments, the devices exhibit little to no leakage under typical blood flow pressures (including high blood flow pressures, e.g., about 175 mm Hg). For example, in some embodiments, a device according to the invention employed within a valved hemodialysis system may exhibit less than about 1 cc fluid leakage after 48 hours when the valve is closed and a flow of liquid is pumped against the device at 175 mm Hg. For example, in some embodiments the device may exhibit less than about 0.8 or less than about 0.5 cc fluid leakage under these conditions. This demonstrates the ability of the devices in some embodiments to provide a reliable seal to prevent blood from entering the device after it has been closed off by the valve of a hemodialysis system. In certain embodiments, the devices can operate effectively at even higher blood flow pressures.

In certain embodiments of the present disclosure, the device has resiliency and rebound strength that exceeds conventional devices. The device may, in certain embodiments, exhibit an opening force of greater than about 200 grams, greater than about 250 grams, or greater than about 260 grams. For instance, in certain embodiments, the device of the present disclosure exhibits an opening force of about 200 to about 300 grams, more particularly from about 250 to about 290 grams, still more particularly from about 260 to about 280 grams.

In certain embodiments, the devices of the present disclosure exhibit the same opening forces when opposing walls of the device are held together for about 48 hours (e.g., under 20+ PSI of pressure). This is relevant in the context of a hemodialysis system, as this time period may replicate the time between dialysis sessions. In this context, it is important to ensure that the device will open up after being pressed shut for 48 hours, again allowing open access to fluid flow for dialysis. In some embodiments, the device exhibits a reopened lumen of at least about 3 mm diameter within 30 seconds after the pressure is removed.

Surprisingly, in certain embodiments, the devices described herein are capable of being repeatedly opened and closed, with no decrease in performance. For example, in certain embodiments, the devices can be fully closed and reopened about 1,000 times or more, about 2,000 times or more, or about 3,000 times or more, without resulting in any observable wear or deformation. For example, in certain embodiments, the device beneficially does not exhibit any significant change in inside or outside dimensions after these closing and reopening cycles. In some embodiments, the deflection/recovery force exhibited by the device does not exhibit any significant change over the course of these closing and reopening cycles. In certain embodiments, the device does not lose any significant amount of particulate matter over the course of the cycles.

Such a capability is advantageous, for example, in hemodialysis, where the creation and maintenance of vascular access is required. In certain embodiments, devices described herein have been subjected to repetitive opening and closing studies and demonstrate, no signs of wear or deformation (e.g., tears, deformation, cracking, delamination, loss of integrity) in a 37° C. (body temperature) water bath after 3,000 opening and closing cycles. In certain embodiments, this data illustrates that devices according to the present disclosure can survive at least about 19 years of dialysis access (based on a calculation of 19 years×3 times per week×52 weeks/year=2,964 cycles of pressure and deflation). This data is significant, given that stents are generally not constructed to undergo repetitive opening and closing processes. The unique design of the devices provided herein allow them to be used effectively in this fashion.

The means by which the composite devices described herein are prepared can vary. Generally, an ePTFE layer is provided, e.g., in the form of a tube (e.g., a biax tube). In certain embodiments, a tubular frame is positioned over the ePTFE layer. The thermoplastic elastomeric component is then applied to the tubular frame. The thermoplastic elastomer (e.g., polyurethane) layer is preferably applied as a tube; however, it could also be applied as a film or sheet. It is noted that the thickness of the thermoplastic elastomeric component is advantageously such that, when melted and flowed, the thermoplastic elastomeric component is present within the device at a sufficient concentration to allow for the desired properties to be achieved (i.e., adhesion between the layers and some degree of penetration of the thermoplastic elastomeric component into one or both of the ePTFE layers). In some embodiments, an additional ePTFE layer is applied over the thermoplastic elastomeric component (e.g., in the form of a second ePTFE biax tube).

The layered structure is then typically heated and/or compressed. The heating and/or compression can, in some embodiments, serve to imbed the frame within the thermoplastic elastomeric component. Advantageously, the heating and/or compressing steps are sufficient to flow the thermoplastic elastomeric layer onto/into one or both adjacent ePTFE layers (e.g., by heating the layered device to an appropriate temperature), allowing the thermoplastic elastomeric component to penetrate the pores therein. The appropriate temperature for the heating would be well understood by one of skill in the art to be that temperature at which the thermoplastic elastomer is slightly melted (and thus, exhibits some degree of “flow”).

The temperature and time selection of the heating step is based on material selection and is important for successful bonding of the composite layers. If there is not enough heat, the thermoplastic elastomer does not melt and adhere to the other layers of the construct, possibly resulting in delamination and thus an inflexible, nondurable composite. If too much heat is applied, bonding will be present but the composite may become brittle and may lack robustness. Too much heating can also result in the undesirable denaturing of the thermoplastic elastomer. When sintering or bonding composite layers it is necessary to ensure that temperatures are selected to properly sinter the material, such that the resulting product has good mechanical integrity, proper adhesion, no delamination of the layers, and no denaturing of the polymer.

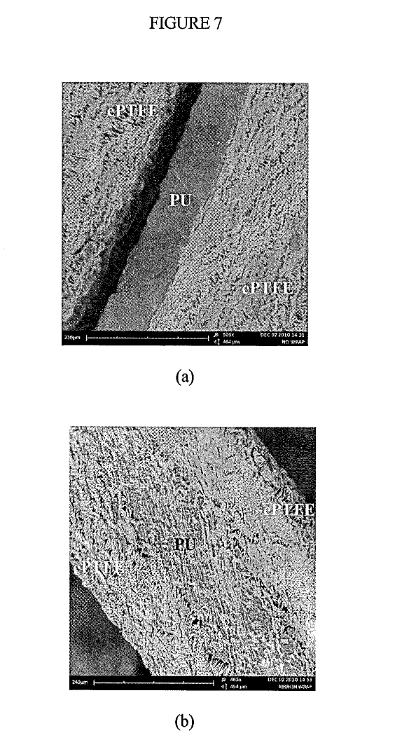

The degree of compression and the nature of accomplishing the compression may vary. Advantageously, in certain embodiments, the compression and heating steps are conducted simultaneously (e.g., by applying a compression wrap to the layered structure and heating the compression-wrapped structure). Varying levels and types of treatment can provide materials of varying quality. FIG. 7 is a comparison of of two ePTFE/PU composite device cross-sections, wherein (a) is a suboptimal device and (b) is a more optimal device.

The present disclosure can be better understood with reference to the following examples, which are not intended to be limiting of the invention.

### EXAMPLES

The following general guidelines are used for the processing examples described herein of various ePTFE and thermoplastic elastomeric composite constructions.

1. A radially expanded fully sintered ePTFE biax tube is placed over a round mandrel base to form the desired tubular geometric shape.

2. The stent frame is then placed over the biax tube.

3. The thermoplastic elastomer (e.g., polyurethane) polymer tube of desired thickness, typically about 0.5 to 1000 μm, is then placed over the biax tube/stent construct to serve as an adhesive as well as an impermeable layer.

4. A second radially expanded fully sintered ePTFE biax tube of the same or different IND and/or thickness is then placed over the biax tube/stent/thermoplastic elastomeric tube construct.

5. A compression wrap is then applied to the final construct and heated at a temperature of about 35° C. to about 485° C. to allow all materials to bond together.

6. Once the heated and compressed composite device is removed from the oven and cooled, the compression wrap is removed and the composite is tested for specified properties.

### Example 1

### ePTFE/frame/PU/ePTFE

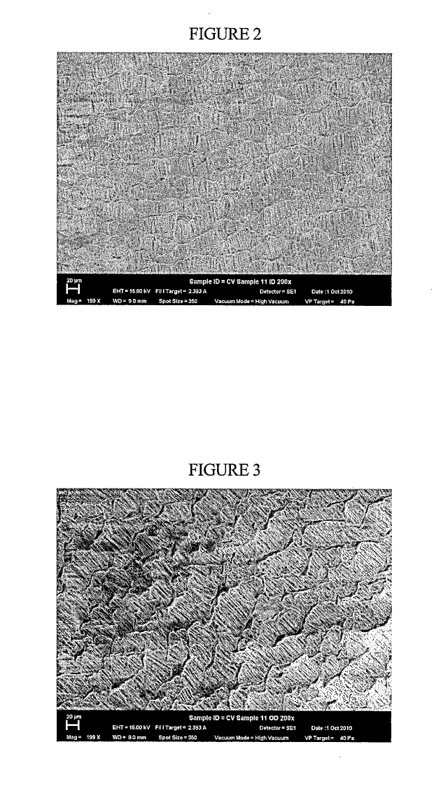

A biaxally (biax) expanded ePTFE tube with an internodal distance (IND) of 30 μm was stretched over a stainless steel rod and placed into an oven at 385° C. for 6 minutes, cooled, and cut into desired lengths. Oriented 6 mm ID tubes with an IND of 20 μm will serve as the ID of the construct (FIG. 2) while oriented 7 mm ID tubes with an IND of 20 μm will serve as the OD of the construct (FIG. 3).

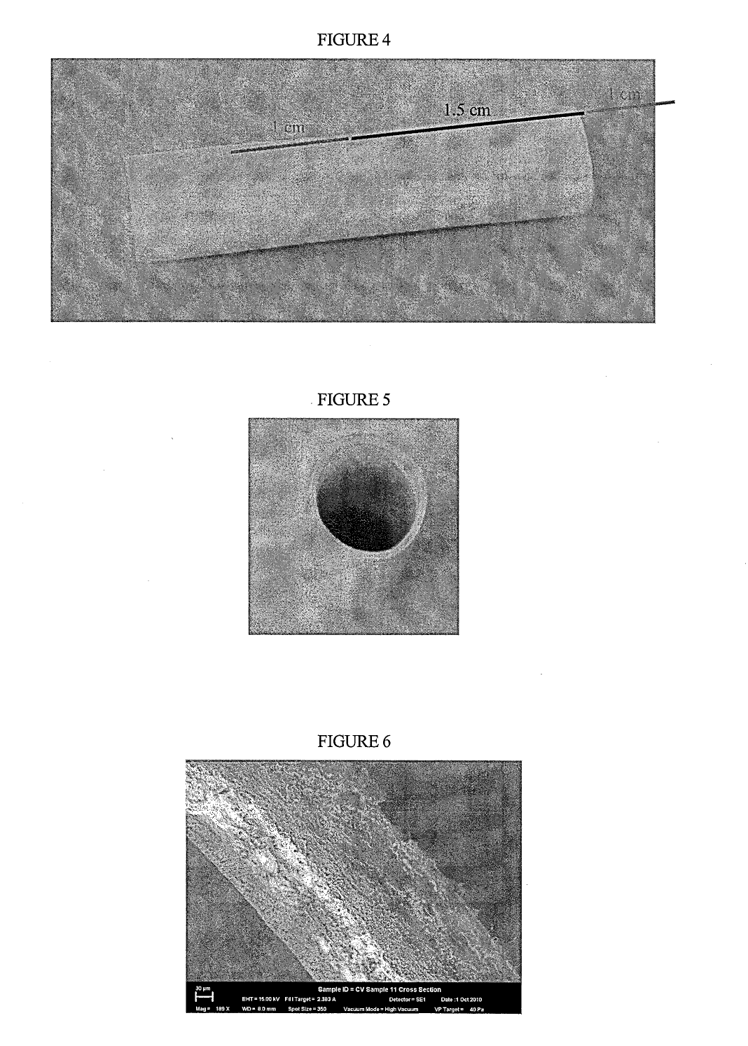

The ID tube was placed over a mandrel, followed by the placement of a stent. A Chronoflex C80A (PU) tube of 6 mm ID was slid over the stent and ID assembly. The OD tube was added and placed over the entire construction. A compression wrap was placed securely over the completed construct and placed in an oven at 240° C. for 8 minutes. The compression wrap was removed and the composite taken off the mandrel (FIGS. 4 and 5). The stent composite was determined to have a 0.35 mm thickness (FIG. 6).

These and other modifications and variations to the present disclosure can be practiced by those of ordinary skill in the art, without departing from the spirit and scope of the present disclosure, which is more particularly set forth in the appended claims. In addition, it should be understood that aspects of the various embodiments can be interchanged both in-whole or in-part. Furthermore, those of ordinary skill in the art will appreciate that the foregoing description is by way of example only, and is not intended to limit the disclosure.

What is claimed is:

1. A composite prosthetic shunt comprising:
an inner lumen;
a first tubular layer of expanded polytetrafluoroethylene (ePTFE) having nodes and fibrils around the inner lumen;
a tubular frame imbedded in polyurethane positioned around and overlying the first tubular layer of ePTFE; and
a second tubular layer of ePTFE having nodes and fibrils positioned around and overlying the tubular frame imbedded in polyurethane;
wherein the polyurethane penetrates at least about 50% of the spaces between the nodes and fibrils of at least one of the first and second tubular layers of ePTFE.

2. The composite prosthetic shunt of claim 1, wherein the average cross-sectional thickness of the shunt wall is between about 0.25 mm and 0.51 mm.

3. The composite prosthetic shunt of claim 1, wherein the shunt walls exhibit an average porosity of less than about 20%.

4. The composite prosthetic shunt of claim 1, wherein the shunt walls exhibit an average porosity of about 0%.

5. The composite prosthetic shunt of claim 1, wherein the polyurethane penetrates at least about 50% of the spaces between the nodes and fibrils of both the first and second tubular layers of ePTFE.

6. The composite prosthetic shunt of claim 1, wherein the polyurethane penetrates at least about 80% of the spaces between the nodes and fibrils of at least one of the first and second tubular layers of ePTFE.

7. The composite prosthetic shunt of claim 1, wherein the polyurethane penetrates at least about 80% of the spaces between the nodes and fibrils of both the first and second tubular layers of ePTFE.

8. The composite prosthetic shunt of claim 1, wherein the shunt exhibits a radial force such that after the shunt is compressed to close the inner lumen for 48 hours, the shunt fully reopens when the compression is removed.

9. The composite prosthetic shunt of claim 1, wherein the shunt exhibits an opening force of greater than about 200 grams.

10. The composite prosthetic shunt of claim 1, wherein the shunt exhibits an opening force of about 200 to about 300 grams.

11. The composite prosthetic shunt of claim 1, wherein the shunt exhibits no substantial decrease in performance after being compressed to close the inner lumen and then opened about 2,000 times or more.

12. The composite prosthetic shunt of claim 1, wherein the shunt exhibits no substantial decrease in performance after being compressed to close the inner lumen and then opened about 3,000 times or more.

13. The composite prosthetic shunt of claim 11, wherein the no substantial decrease in performance is evidenced by one or more of: no significant change in inside or outside dimensions of the shunt; no observable wear or deformation; no significant change in the recovery force of the shunt; and no significant loss of particulate material from the shunt.

14. The composite prosthetic shunt of claim 12, wherein the no substantial decrease in performance is evidenced by one or more of: no significant change in inside or outside dimensions of the shunt; no observable wear or deformation; no significant change in the recovery force of the shunt; and no significant loss of particulate material from the shunt.

15. A hemoaccess valve system comprising the composite prosthetic shunt of claim 1.

16. A method for making a composite prosthetic shunt, comprising:
applying a polyurethane sheet or tube to a construct comprising a tubular frame overlying a first ePTFE tubular structure;
applying a second ePTFE tubular structure overlying polyurethane sheet or tube to form a layered composite;
compressing the layered composite; and
heating the layered composite such that the polyurethane penetrates at least about 50% of the spaces between the nodes and fibrils of at least one of the first and second tubular layers of ePTFE.

17. The method of claim 16, wherein the compressing and heating steps are conducted at the same time.

18. The method of claim 16, wherein the heating is conducted at a temperature at or above the melting temperature of the polyurethane.

19. The method of claim 16, wherein the compressing step comprises wrapping the layered composite with a compression wrap.
